(19) United States
(12) Patent Application Publication
(10) Pub. No.: US 2020/0069925 A1
(43) Pub. Date: Mar. 5, 2020

(54) VENOUS-TO-VENOUS LOCO-REGIONAL DELIVERY OF A THERAPEUTIC AGENT

(71) Applicant: Reynaldo Calderon, Houston, TX (US)

(72) Inventor: Reynaldo Calderon, Houston, TX (US)

(21) Appl. No.: 16/677,965

(22) Filed: Nov. 8, 2019

Related U.S. Application Data
(63) Continuation-in-part of application No. 16/363,520, filed on Mar. 25, 2019, now abandoned.
(60) Provisional application No. 62/647,075, filed on Mar. 23, 2018.

(51) Int. Cl. A61M 25/10; A61M 25/00; A61M 25/06; A61M 5/145; A61M 5/00; A61M 1/00

(52) U.S. Cl. CPC A61M 25/1011; A61M 25/0026; A61M 25/0662; A61M 2025/105; A61M 5/007; A61M 1/0084; A61M 2025/0002; A61M 5/14546

(57) ABSTRACT

The methods, systems, and devices disclosed herein generally involve a venous-to-venous delivery of drugs to a targeted region within a patient. A triple lumen catheter device is disclosed that is particularly suitable for delivery of therapeutic drugs via a closed loop, preventing the circulation of toxic agents throughout the body, thereby creating an isolated treatment zone that maximizes local therapeutic concentrations while preventing agents from escaping to the rest of the body.

### FIELD

The present invention relates generally to methods and systems for venous-to-venous closed-loop delivery of a therapeutic agent, and in a particular though non-limiting embodiment to a method and system for venous-to-venous closed-loop delivery of a therapeutic agent using a triple-lumen catheter comprising a distal balloon catheter for infusion, a central sheath for monitoring pressures, and a proximal balloon catheter for aspiration.

### BACKGROUND

Many targeted approaches to chemotherapy exist. However, all have an arterial approach that fails to prevent systemic leakage. For example, anticancer agents delivered by standard catheters flow directly into an artery and travel uncontrolled to all areas of the body. This results in inadequate therapeutic uptake while causing damaging side-effects. There is, therefore, an unmet need for a venous approach to address this problem.

Consequently, new methods are needed to disrupt the physiologically challenging hemodynamics of hepatic tumor vasculature and microcirculation to optimize drug penetration, duration of exposure and systemic toxicity reduction.

### SUMMARY

The method disclosed herein comprises introduction of a venous-to-venous closed-loop delivery of a therapeutic agent by employing a triple-lumen catheter, further comprising a distal balloon catheter for infusion, a central sheath for monitoring pressures and a proximal balloon catheter for aspiration.

In one specific though non-limiting embodiment, the catheter delivers a contrast agent via a major hepatic vein to a target liver site; monitors key pressure and flow variables over time; and retrieves the unabsorbed CT agent to minimize systemic toxicity. A syringe infusion pump, withdrawal pump, and pressure transducer control the flow rates and pressures.

FIG. 1 illustrates a compelling 3-D rendering of the dual circuits, confirming the viability of the design in controlling pressure and flow dynamics in the hepatic circulation.

In another example embodiment, the catheter balloon distends, thereby slowing the circulation of the CT contrast agent to reveal the vascular or lymphatic composition of the tumor. In yet another embodiment, the catheter infuses, monitors pressure and aspirates a therapeutic agent at a controllable rate.

### BRIEF DESCRIPTION OF THE DRAWING

The patent or application file contains at least one drawing executed in color. Copies of this patent or patent application publication with color drawing(s) will be provided by the Office upon request and payment of the necessary fee.

The aforementioned features and advantages of the disclosed technology as well as additional features and advantages thereof will be more clearly understood hereinafter as a result of a detailed description of preferred implementations when taken in conjunction with the drawings.

FIG. 1 illustrates a 3-D rendering of the dual circuits, confirming the viability of the design in controlling pressure and flow dynamics in the hepatic circulation.

FIG. 2 illustrates the infusion pump and the withdrawal pump while also signaling the closed loop pathway in which the therapeutic agents enter and exit.

FIG. 3a illustrates a localized pathway in which the therapeutic agent enters and exists with the use of the triple lumen catheter.

FIG. 3b illustrates a systemic delivery of the drugs circulating the whole body as opposed to being localized.

FIG. 4 illustrates the infusion catheter which pushes medicine into the body at point 1. Point A shows the distal balloon inflated to occlude the vein and direct the countervailing flow of drugs while the central catheter at point 2 monitors pressure and keeps the vein open. Point 3 demonstrates the suction catheter which pulls medicine out of the body. Lastly, point B shows a balloon occludes the vein and prevents medicine from escaping to other parts of the body.

### DESCRIPTION OF EXAMPLE EMBODIMENTS

The disclosed method comprises a venous-to-venous loco-regional delivery of a therapeutic agent using a novel catheter-based delivery system. Certain exemplary embodiments will now be described to provide an overall understanding of the principles of the structure, function, and use of the methods, systems, and devices disclosed herein. One or more examples of these embodiments are illustrated in the accompanying drawings. Those skilled in the art will understand that the methods, systems, and devices specifically described herein and illustrated in the accompanying drawings are non-limiting exemplary embodiments and that the scope of the present invention is defined solely by the claims.

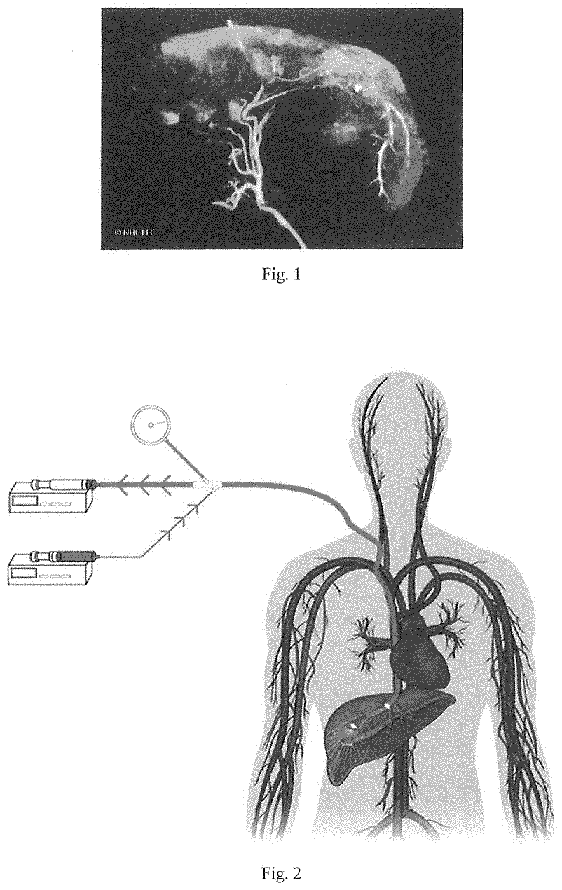

FIG. 1 illustrates a compelling 3-D rendering of the dual circuits, confirming the viability of the design in controlling pressure and flow dynamics in the hepatic circulation. Ordinarily skilled artisans will readily appreciate that further embodiments of the disclosed method will deliver a plurality of therapeutic agents, e.g. a hepatocellular carcinoma drug or saline.

FIG. 2 is a schematic illustration of the main hepatic vein and the general pathway for delivery of therapeutic agents. Under image guidance an interventional radiologist can guide a catheter through the body directly to a tumor in the liver as illustrated.

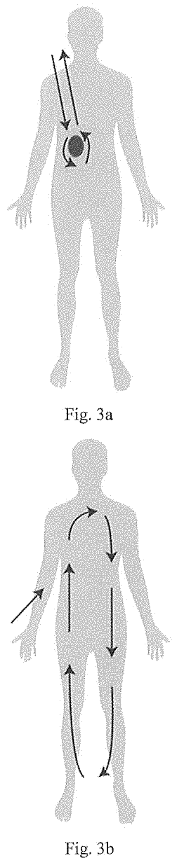

FIGS. 3a and 3b further illustrates the targeted pathway for delivery in comparison to a systemic delivery of therapeutic agents. The targeted approach is less damaging to the patient as it reduces the amount of systemic leakage and other adverse side effects that affect the overall quality of life for the patient as well as the ability to tolerate further treatment.

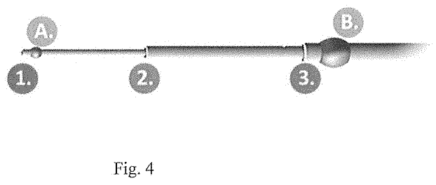

With example arrangement illustrated in FIG. 4, the catheter consists of one catheter that infuses, one that withdraws, and one that monitors pressure. This arrangement allows the targeted approach previously mentioned.

The claimed invention has been depicted and described in detail above with respect to several exemplary embodiments, ordinarily skilled artisans in the relevant fields will readily appreciate that minor changes to the description, and various other modifications, omissions and additions may also be made without departing from either the spirit or scope thereof.

1. A system for venous-to-venous closed-loop delivery of a therapeutic agent, said method comprising:
a triple lumen catheter for delivering said therapeutic agent, wherein said triple lumen catheter further comprises a distal balloon catheter for infusion; and
a central sheath for monitoring pressures; and a proximal balloon catheter for aspiration.

2. The system of claim 1, wherein the infusion catheter is in connection with the distal balloon.

3. The system of claim 1, wherein the proximal balloon suctions unused drug out of the body.

4. The system of claim 1, further comprising a transducer to control flow rates and pressures.

5. A method of venous-to-venous closed-loop delivery of a therapeutic agent, the method comprising:
positioning a triple lumen catheter into but not limited to a major hepatic vein;
targeting treatment through a closed loop delivery; and
inserting a specified amount of drug and retrieving unused drug.

6. The method of claim 5, further comprising delivering via a major vein and retrieving unused drug through same major vein.

7. The method of claim 5, further comprising monitoring flow rates and pressures of infused drug.

8. The method of claim 5, further comprising minimizing toxicity level by retrieval of unused drug.
